(19) United States
(12) Patent Application Publication
(10) Pub. No.: US 2022/0265684 A1
(43) Pub. Date: Aug. 25, 2022

(54) COMPOSITIONS AND METHODS FOR TREATING ULCERATIVE COLITIS

(71) Applicant: 9 METERS BIOPHARMA, INC., Raleigh, NC (US)

(72) Inventors: Nir BARAK, Tel Aviv (IL); Sireesh APPAJOSYULA, Raleigh, NC (US); Patrick H. GRIFFIN, Raleigh, NC (US)

(21) Appl. No.: 17/392,483

(22) Filed: Aug. 3, 2021

Related U.S. Application Data
(60) Provisional application No. 63/153,139, filed on Feb. 24, 2021.

(51) Int. Cl. A61K 31/655

(52) U.S. Cl. CPC A61K 31/655; A61K 9/0053

(57) ABSTRACT

The invention, in various aspects and embodiments, provides compositions and methods for treating or managing ulcerative colitis (UC) (including mild or moderate UC), including intestinal and extraintestinal symptoms, including but not limited to arthritis. The compositions and methods in various embodiments relate to oral administration of pharmaceutical compositions that comprise an effective amount of one or more agents that forms at least one 4-APAA compound by azo reduction in the colon. In various embodiments, active agent is available for local action in the lumen of the large intestine. Further, as described herein, a significant portion of the 4-APAA formed by azo reduction in the large intestine is systemically available to ameliorate extraintestinal symptoms.

### BACKGROUND

Ulcerative colitis is a chronic inflammatory disease of complex etiology that affects various portions of the gastrointestinal (GI) tract, particularly the lower GI tract, and more particularly the colon and/or rectum. Some UC patients experience extraintestinal inflammatory complications, such as arthritis, or inflammation (pain with swelling) of the joints, which is reported to occur in about 21% of patients with ulcerative colitis. Compositions and methods for managing the intestinal and extraintestinal symptoms of UC are needed.

### DETAILED DESCRIPTION

The invention, in various aspects and embodiments, provides compositions and methods for treating or managing ulcerative colitis (UC) (including mild or moderate UC), including intestinal and extraintestinal symptoms, including but not limited to arthritis. The compositions and methods in various embodiments relate to oral administration of pharmaceutical compositions that comprise an effective amount of one or more agents that form at least one 4-APAA compound by azo reduction in the colon. In various embodiments, active agent is available for local action in the lumen of the large intestine. Further, as described herein, a significant portion of the 4-APAA formed by azo reduction in the large intestine is systemically available to ameliorate extraintestinal symptoms.

In one aspect, this disclosure provides a method for treating ulcerative colitis in a subject with extraintestinal inflammatory complications. The method comprises orally administering a pharmaceutical composition comprising an effective amount of one or more agents that form 4-APAA by azo reduction. In various embodiments, the subject has extraintestinal complications of ulcerative colitis selected from inflammation of one or more organs or tissues other than the lower gastrointestinal tract. Such tissues and organs include joints, skin, liver, kidneys, pancreas, bone, eyes, and mouth.

In some embodiments, the subject has arthritis associated with ulcerative colitis. The arthritis associated with ulcerative colitis may be peripheral arthritis, axial arthritis, and/or ankylosing spondylitis. In the general population, individuals with peripheral arthritis may use nonsteroidal anti-inflammatory drugs (NSAIDs) to reduce pain and swelling of the joints. However, as a rule, these medications—which include aspirin and ibuprofen—are not a good option for many with UC because they can irritate the intestinal lining and exacerbate the disease.

Peripheral arthritis usually affects the large joints of the arms and legs, including the elbows, wrists, knees, and ankles. The discomfort may be “migratory,” moving from one joint to another. If left untreated, the pain may last from a few days to several weeks. Peripheral arthritis tends to be more common among people who have ulcerative colitis or Crohn's disease of the colon. Although no specific test can make an absolute diagnosis, various diagnostic methods—including analysis of joint fluid, blood tests, and X-rays—are used to rule out other causes of joint pain.

Axial arthritis, also known as spondylitis or spondyloarthropathy, produces pain and stiffness in the lower spine and sacroiliac joints (at the bottom of the back). These symptoms may come on months or even years before the symptoms of IBD appear. Unlike peripheral arthritis, axial arthritis may cause permanent damage if the bones of the vertebral column fuse together—thereby creating decreased range of motion in the back. In some cases, a restriction in rib motion may make it difficult for people to take deep breaths.

A more severe form of spinal arthritis, ankylosing spondylitis (AS), is a rare complication, affecting between 2% and 3% of people with IBD. In addition to causing arthritis of the spine and sacroiliac joints, ankylosing spondylitis can cause inflammation of the eyes, lungs, and heart valves. Occasionally, AS foretells the development of IBD. Ankylosing spondylitis typically strikes people under the age of 30, mainly adolescents and young adult males, appearing first as a dramatic loss of flexibility in the lower spine.

In other embodiments, the subject has extraintestinal inflammatory conditions of the skin (e.g., rash, erythema nodosum, or pyroderma gangrenosum), mouth (e.g, stomatitis), bone (e.g., osteoporosis, osteopenia, or osteomalacia), eyes (e.g., uveitis, keratopathy, episcleritis, or dry eye), kidneys (e.g., amyloidosis or glomerulonephritis), liver (e.g., hepatic steatosis, hepatitis, or primary sclerosing cholangitis), and pancreas (e.g., pancreatitis).

The methods and compositions described herein involve agents that form 4-APAA by azo reduction. U.S. Pat. Nos. 6,903,082, 7,425,578, and 8,048,924, which are hereby incorporated by reference in their entireties, describe compounds in which 5-ASA compounds are conjugated via an azo bond to 4-aminophenylacetic acid (4-APAA) compounds (referred to herein as NM-004). The azo bond can be cleaved by enzymes secreted by bacteria in the lower gastrointestinal tract, yielding pharmaceutically active 5-ASA and 4-APAA compounds. Studies indicate that 5-ASA and 4-APAA compounds inhibit intestinal inflammation by different mechanisms and can have synergistic effects when administered together in a compound such as NM-004.

If given orally as separate compounds, 5-ASA and 4-APAA are rapidly absorbed and never achieve high concentrations in the lumen of the colon. In NM-004, sodium salts of 5-ASA and 4-APAA are chemically coupled through an azo bond, and as a result, NM-004 is poorly absorbed in the small intestine. However, upon reaching the colon, azo reductases produced by colonic bacteria cleave the azo bond releasing both moieties (5-ASA and 4-APAA), and associated metabolites [acetylated 5-ASA (5-ace-ASA) and acetylated 4-APAA (4-ace-APAA)] will be produced. High local concentrations of 5-ASA and 4-APAA delivered through NM-004 are then beneficial as local treatment of ulcerative colitis. Further, pharmacokinetic studies shown here, demonstrate that, although the majority of 5-ASA and 4-APAA are active in the colon, there is a systemic absorption especially of 4-APAA, and particularly the active metabolite 4-ace-APAA. Accordingly, these compounds producing 4-APAA by azo reduction are well suited for treating UC having extraintestinal inflammatory symptoms.

In accordance with this disclosure, the compositions comprising one or more agents that produces 4-APAA compounds by azo reduction, are delivered to the lower gastrointestinal tract. The term “lower gastrointestinal tract” as used herein includes the lower part of the small intestine, the rectum, and the large intestine (colon) and/or ileo-anal pouch, if present. In some embodiments, the composition delivers the agent to at least the colon.

In some embodiments, the agent is a compound of the formula:

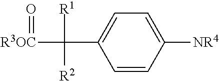

where R¹, R², and R³ are independently hydrogen, halogen, or C1 to C4 alkyl, and R⁴ is:

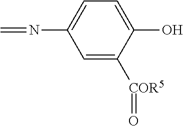

where R⁵ is selected from hydrogen, halogen, and C1 to C4 alkyl; or R⁴ is:

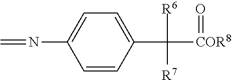

where R⁶, R⁷, and R⁸ are independently hydrogen, halogen, or C1 to C4 alkyl. For example, in some embodiments, the compound is a 4-aminophenylacetic acid azo-bonded dimer. In some embodiments, the compound is 5-(4-carboxymethyl-phenylazo)-2-hydroxy-benzoic acid.

The term “4-APAA compounds” as used herein includes 4-aminophenylacetic acid and related compounds, including 4-aminophenylacetic acid (4-APAA):

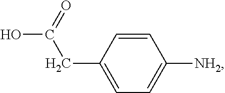 [SMILES: Nc1ccc(CC(=O)O)cc1]

as well as (4-acetylaminophenyl)-acetic acid (actarit):

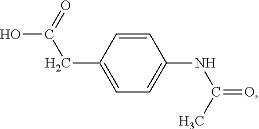

and other 4-aminophenylacetic acid derivatives.

In some embodiments, the agent is an azo-bonded dimer that, in addition to forming a 4-APAA compound, forms a 5-ASA compound. The term “5-ASA compound” as used herein includes 5-aminosalicylic acid (mesalamine) and related compounds, including compounds that react under physiological conditions to form or release 5-ASA and related compounds.

In exemplary embodiments, the agent that forms 4-APAA by azo reduction is a 4-aminophenylacetic acid azo bonded dimer [4-(4-carboxymethyl-phenylazo)-phenyl]-acetic acid:

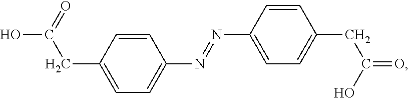 [SMILES: O=C(O)Cc1ccc(/N=N/c2ccc(CC(=O)O)cc2)cc1]

or is 5-(4-carboxymethyl-phenylazo)-2-hydroxybenzoic acid:

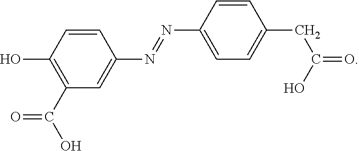 [SMILES: O=C(O)Cc1ccc(/N=N/c2ccc(O)c(C(=O)O)c2)cc1]

In some embodiments, the pharmaceutical composition, in addition to comprising an effective amount of an agent that forms 4-APAA by azo reduction, may also include non-azo bonded 5-ASA or 4-APAA. In these embodiments, higher levels of these agents will be systemically available for extraintestinal symptoms. In these or other embodiments, the composition may further comprise azo-bonded 5-ASA dimer, or azo-bonded 4-APAA dimer. In these embodiments, the composition can control the ratios of 5-ASA and 4-APAA available in the colon and/or for systemic absorption from the colon. In various embodiments, the ratio of azo-bonded 4-APAA to azo-bonded 5-ASA is more than about 1:1, so that the amount of 4-APAA is sufficient to control intestinal and extraintestinal symptoms. For example, the ratio may be about 2:1, about 3:1, about 4:1, or about 5:1. In some embodiments, the ratio of azo-bonded 5-ASA is higher than azo-bonded 4-APAA, such as about 2:1, about 3:1, about 4:1, or about 5:1. These embodiments can modfy the levels of the agents to leverage the distinct mechanisms of action for therapeutic benefit.

The term “related compounds” as used herein includes analogs, derivatives, and compounds comprising the basic structural feature that are responsible for a compound's therapeutic activity. For all compounds described herein, the invention includes the esters or pharmaceutically acceptable salts of the compounds.

The composition may include pharmaceutically acceptable salts of the agent(s). Pharmaceutically acceptable salts retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, liimaric acid, gluconic acid, citric acid, malic acid, ascorbic acid, benzoic acid, talmic acid, palmitic acid, alginic acid, poly-glutamic acid, naphthalenesulfonic acid, methanesulfonic acid, p-toluenesulfonic acid, naplithalenedisulfonic acid, polygalacturonic acid, and the like; and (b) salts formed from elemental anions such as chlorine, bromine, and iodine or cations such as sodium and potassium.

The composition may include other pharmaceutically acceptable components, generally formulated for oral administration. The term “pharmaceutically acceptable components” include salts, carriers, excipients and/or diluents, and is a component that (i) is compatible with the other ingredients of the composition in that it can be combined with active pharmaceutical ingredients, without rendering the active pharmaceutical ingredients unsuitable for its intended purpose, and (ii) is suitable for use with subjects without undue adverse side effects (such as toxicity, irritation, and allergic response). Side effects are “undue” when their risk outweighs the benefit provided by the pharmaceutical composition.

In accordance with embodiments of the invention, the composition is administered at a dose of active agent (including the presence of azo-bonded and non azo-bonded agents) and schedule so as to provide sufficient 4-Ace-APAA to manage extraintestinal inflammatory symptoms. For example, in some embodiments, the agent is administered at a dose and schedule that achieves an AUC of at least about 5 μg*hr/mL for 4-Ace-APAA, or at least about 10 μg*hr/mL for 4-Ace-APAA, or at least about 15 μg*hr/mL for 4-Ace-APAA. Alternatively or in addition, the agent is administered at a dose and schedule that achieve Cmax of at least about 0.5 μg/mL for 4-Ace-APAA, or least about 1.0 μg/mL for 4-Ace-APAA, or at least about 1.5 μg//mL for 4-Ace-APAA.

For example, the agent may be administered at a daily dose of at least about 500 mg, or at least about 1000 mg, or at least about 2000 mg, or at least about 3000 mg, or at least about 4000 mg. In some embodiments, the agent is administered at a daily dose of from about 500 mg to about 5000 mg, or a daily dose of from about 1000 mg to about 4000 mg, or a daily dose of from about 2000 mg to about 4000 mg. In some embodiments, the daily dose is about 3000 mg, about 3500 mg, or about 4000 mg. The daily dose may be administered as a single dose, or a plurality of sub-doses. In various embodiments, the agent is administered once daily, twice daily, or three times daily. In some embodiments, the daily dose is administered for at least two weeks, or at least four weeks, or at least eight weeks, or more.

In other aspects, the invention provides a pharmaceutical composition for oral administration, the composition comprising an amount of one or more agents that form 4-APAA by azo reduction to manage both intestinal and extraintestinal symptoms of ulcerative colitis. For example, in various embodiments, the composition comprises at least about 500 mg of the agent(s) (including the total amount of both azo-bonded and non azo-bonded agents in the composition). The agent(s) can be as described, including a 4-aminophenylacetic acid azo bonded dimer (forming 4-APAA upon azo reduction) or 5-(4-carboxymethyl-phenylazo)-2-hydroxy-benzoic acid (forming 4-APAA and 5-ASA upon azo reduction). In various embodiments, the composition is a unit dose as described, including unit doses of at least about 750 mg, or at least about 1000 mg of the agent(s), or at least about 2000 mg of the agent(s). Exemplary unit doses for oral administration include about 750 mg, about 1000 mg, about 1250 mg, about 1500 mg, about 1750 mg, and about 2000 mg. Compositions suitable for oral administration may be presented as discrete units such as capsules, cachets, tablets, or lozenges, each containing a predetermined amount of a compound of the present invention as a powder or granules; or a suspension in an aqueous liquor or a non-aqueous liquid, such as a syrup, an elixir, or an emulsion.

The terms “treat” or “treating” as used herein refers to any type of treatment that imparts a modulatory effect, which, for example, can be a beneficial effect to a subject afflicted UC, including extraintestinal inflammatory symptoms, including improvement in the condition of the subject, such as a reduction in inflammatory symptoms, and slowing of disease progression.

It is emphasized that the terms “comprises” and “comprising”, when used in this application, specify the presence of stated features, steps, or components and do not preclude the presence or addition of one or more other features, steps, components, or groups thereof. The singular forms “a,” “an,” and “the” are intended to include the plural forms as well, unless the context clearly indicates otherwise.

The term “about” means ±10% of the associated numerical value.

### EXAMPLES

### Example 1: Systemic Absorption of 4-APAA for Treatment of Ulcerative Colitis Associated with Arthritis

The anti-inflammatory and immunomodulatory properties of 5-ASA and 4-APAA, respectively, were combined to create “NM-004” for the treatment of mild to moderate ulcerative colitis (UC) associated with arthritis. NM-004 consists of 5-ASA, which has anti-inflammatory activity, and 4-APAA, which has immunomodulatory activity, chemically coupled through an azo bond. If given orally as separate compounds, 5-ASA and 4-APAA are rapidly absorbed and never achieve high concentrations in the lumen of the colon. In NM-004, sodium salts of 5-ASA and 4-APAA are chemically coupled through an azo bond, and as a result, NM-004 is poorly absorbed in the small intestine. However, upon reaching the colon, azo reductases produced by colonic bacteria cleave the azo bond releasing both active moieties (5-ASA and 4-APAA) and associated metabolites [acetylated 5-ASA (5-ace-ASA) and acetylated 4-APAA (4-ace-APAA)]. Acetylated 4-APAA is the active form, while acetylated 5-ASA is inactive. High local concentrations of 5-ASA and 4-APAA (and their metabolites) delivered through NM-004 are then beneficial as local treatment of ulcerative colitis. Moreover, 4-APAA plus 5-ASA, by its anti-inflammatory and immunomodulatory effects, have benefits over and above those of conventional 5-ASA and immunomodulator drugs that are used for ulcerative colitis.

For example, pharmacokinetic studies show that although the majority of 5-ASA and 4-APAA are active in the colon, there is a systemic absorption, especially of the acetylated metabolite of 4-APAA. In a 14 days repeat dose (1000 mg TID of NM-004) administration study in adult patients with mild to moderate ulcerative colitis, a Cₘₐₓ of 1.29 μg/mL and AUC₀₋ₜ of 11.99 μg*hr/mL was observed for 4-Ace-APAA. In comparison, the Cₘₐₓ of 4-APAA after administration of 100 mg (1 tablet) is 2.24 μg/mL and the AUC_{0-∞} is 4.56 μg*hr/mL [Actarit PI].

In these experiments, the rate of absorption (i.e., peak exposure) is assessed by measuring the peak drug concentration (Cₘₐₓ). The extent of absorption (i.e., total exposure) is the area under the plasma/serum/blood concentration-time curve from time zero to time t, which is AUC₀₋ₜ, where t is the last time point with a measurable concentration. The area under the plasma/serum/blood concentration-time curve from time zero to time infinity is AUC_{0-∞}.

TABLE 1

| | Cmax | AUC0-∞ | |
|---|---|---|---|
| | (μg/mL) | (μg*hr/mL) | |
| NM004 | 0.39 | BLQ | 5-ASA and 4-APAA |
| | | | azo-bonded combination |
| 5-ASA | 0.42 | BLQ | Active form 5-ASA |
| 5-Ace-ASA | 0.44 | BLQ | Inactive form of 5-ASA |
| 4-APAA | 0.40 | BLQ | ProDrug of Actarit |
| 4-Ace-APAA | 1.29 | 11.99 | Active form of APAA (Actarit) |

The demonstrated systemic absorption of 4-APAA supports an unexpected benefit of NM-004 in cases of ulcerative colitis associated with systemic complications, such as arthritis.

1-44. (canceled)

45. A method for treating ulcerative colitis and associated axial arthritis in a subject in need thereof, the method comprising orally administering an effective amount of a pharmaceutical composition comprising a prodrug that is 5-(4-carboxymethyl-phenylazo)-2-hydroxybenzoic acid) that forms 4-acetylaminophenyl-acetic acid (4-Ace-APAA) and 5-aminosalicylic acid (5-ASA) by azo reduction in the large intestine to the subject having ulcerative colitis and associated axial arthritis.

46. The method of claim 45, wherein the pharmaceutical composition further comprises [4-(4-carboxymethyl-phenylazo)-phenyl]-acetic acid or pharmaceutically acceptable salt thereof.

47. The method of claim 46, wherein the ratio of azo-bonded 4-aminophenylacetic acid (4-APAA) to azo-bonded 5-ASA in the composition is more than 1:1.

48. The method of claim 47, wherein the ratio of azo-bonded 4-aminophenylacetic acid (4-APAA) to azo-bonded 5-ASA in the composition is more than 2:1.

49. The method of claim 45, wherein the effective amount is a daily dose of at least 500 mg of prodrug.

50. The method of claim 45, wherein the effective amount is a daily dose of at least 1000 mg of prodrug.

51. The method of claim 50, wherein the effective amount is a daily dose of from 1000 mg to 4000 mg of the prodrug.

52. The method of claim 51, wherein the composition is administered twice daily or three times daily.

53. The method of claim 52, wherein a daily dose is administered for at least two weeks.

54. The method of claim 45, wherein the composition is administered at a dose and schedule that achieves AUC of at least about 5 μg*hr/mL for 4-Ace-APAA in blood, serum, or plasma.

55. The method of claim 54, wherein the composition is administered at a dose and schedule that achieves AUC of at least about 10 μg*hr/mL for 4-Ace-APAA in blood, serum, or plasma.

56. The method of claim 54, wherein the composition is administered at a dose and schedule that achieves AUC of at least about 15 μg*hr/mL for 4-Ace-APAA in blood, serum, or plasma.

57. The method of claim 53, wherein the composition is administered at a dose and schedule that achieves Cmax in blood, serum, or plasma of at least about 0.5 μg/mL for 4-Ace-APAA.

58. The method of claim 57, wherein the composition is administered at a dose and schedule that achieves Cmax in blood, serum, or plasma of at least about 1.0 μg/mL for 4-Ace-APAA.

59. The method of claim 57, wherein the composition is administered at a dose and schedule that achieves Cmax in blood, serum, or plasma of at least about 1.5 μg/mL for 4-Ace-APAA.

60. The method of claim 45, wherein the ulcerative colitis is mild to moderate.

61. The method of claim 52, wherein a daily dose is administered for at least four weeks.

62. The method of claim 52, wherein a daily dose is administered for at least eight weeks.
